(19) United States
(12) Patent Application Publication
(10) Pub. No.: US 2026/0275225 A1
(43) Pub. Date: Sep. 17, 2026

(54) LIQUID ALKYLATED PHENYL-ALPHA-NAPHTHYLAMINE WITH REDUCED AQUATIC TOXICITY

(71) Applicant: BASF SE, Ludwigshafen am Rhein (DE)

(72) Inventors: Graciela SANCHEZ JIMENEZ, Puebla (MX); Kevin J. DESANTIS, Tarrytown, NY (US); Thomas SCHUSTER, Ludwigshafen am Rhein (DE)

(21) Appl. No.: 19/166,915

(22) PCT Filed: Mar. 13, 2024
(86) PCT No.: PCT/EP2024/056687

§ 371 Date: Sep. 19, 2025

(30) Foreign Application Priority Data
Mar. 20, 2023 (EP) 23162823.1

(51) Int. Cl. C10M 133/54; C10N 70/00

(52) U.S. Cl. CPC C10M 133/54; C10M 2215/067; C10N 2070/00

(57) ABSTRACT

The present invention relates to a method for manufacturing a liquid additive comprising an amine of the formula (1) where the method comprises the steps of alkylating phenyl-alpha-naphthylamine with an olefin selected from linear or branched C8-C12 alkenes to obtain a first raw product comprising the amine of the formula (1); distilling off unreacted olefin from the first raw product to obtain a second raw product; and distilling off unreacted phenyl-alpha-naphthylamine from the second raw product to obtain the liquid additive. The invention also relates to a liquid additive comprising an amine of the formula (1) and phenyl-alpha-naphthylamine, where the liquid additive comprises at least 95 wt % of the amine of the formula (1), and where the concentration of the phenyl-alpha-naphthylamine in the liquid additive is below 0.2 wt %, preferably below 0.1 wt %. It further relates to a use of the liquid additive as in lubricants.

The present invention relates to a method for manufacturing a liquid additive comprising an amine of the formula (1) where the method comprises the steps of alkylating phenyl-alpha-naphthylamine with an olefin selected from linear or branched C₈-C₁₂ alkenes to obtain a first raw product comprising the amine of the formula (1); distilling off unreacted olefin from the first raw product to obtain a second raw product; and distilling off unreacted phenyl-alpha-naphthylamine from the second raw product to obtain the liquid additive. The invention also relates to a liquid additive comprising an amine of the formula (1) and phenyl-alpha-naphthylamine, where the liquid additive comprises at least 95 wt % of the amine of the formula (1), and where the concentration of the phenyl-alpha-naphthylamine in the liquid additive is below 0.2 wt %, preferably below 0.1 wt %. It further relates to a use of the liquid additive as in lubricants.

Alkylated phenyl-alpha-naphthylamines (also known as alkylated PANA) are important additives in lubricants, e.g. for stabilization against oxidation. Many of the alkylated PANA are solid additives, but there are also liquid alkylated PANA known, which are of commercial interest.

Lubricants are needed also in or near aquatic environments, such as on ships or in machines near lakes and rivers. As precaution a low aquatic toxicity of the lubricant and its additives are desirable and often required by law.

Object of the present invention was to find liquid alkylated PANA, which have a reduced aquatic toxicity.

The object was solved by a method for manufacturing a liquid additive comprising an amine of the formula (1)

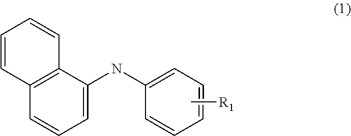

where R₁ is a linear or branched C₈-C₁₂ alkyl, and
where the method comprises the steps of
a) alkylating phenyl-alpha-naphthylamine with an olefin selected from linear or branched C₈-C₁₂ alkenes to obtain a first raw product comprising the amine of the formula (1);
b) distilling off unreacted olefin from the first raw product to obtain a second raw product; and
c) distilling off unreacted phenyl-alpha-naphthylamine from the second raw product to obtain the liquid additive.

The residue R¹ is a linear or branched C₈-C₁₂ alkyl, such as a linear or branched C₈, C₉, C₁₀, C₁₁, or C₁₂ alkyl. Mixtures of linear and branched C₈-C₁₂ alkyls are also possible.

Preferably, R¹ is a linear or branched C₉ alkyl, such as n-nonyl, 1-methyloctyl, 1,1,3-trimethylhexyl, 1,1,5-trimethylhexyl, 1,1,3,4-tetramethylpentyl, 1,1,2,2-tetramethylpentyl, or 1,1,2,4-tetramethylpentyl. Mixtures of linear and branched C₉ alkyls are also possible.

The term “alkyl” usually means in regard to R1 an saturated hydrocarbon group consisting only of hydrogen and carbon atoms.

The residue R¹ can be in ortho, meta, or para position of the phenyl group in the formula (1), where the para position is preferred.

The liquid additive may comprise at least 70, 80, 90, 93, 95, 96, 97, 98, 99, 99.5, or 99.8 wt % of the amine of the formula (1). Preferably, the liquid additive comprises at least 95 wt % of the amine of the formula (1).

Typically, hydrogen atoms are not always displayed in chemical formula. Accordingly, the amine of the formula (1) can also be illustrated by the corresponding following formula:

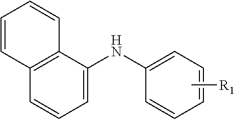

The liquid additive may comprise the phenyl-alpha-naphthylamine, and the concentration of the phenyl-alpha-naphthylamine in the liquid additive can be below 0.2 wt %, preferably below 0.1 wt %. The concentration of the phenyl-alpha-naphthylamine in the liquid additive can be below 0.2, 0.17, 0.15, 0.12, 0.10 or 0.09 wt %. The concentration of the phenyl-alpha-naphthylamine in the liquid additive can be 0.00001 to 0.2 wt %, 0.00001 to 0.17, 0.00001 to 0.15, 0.00001 to 0.12, 0.0001 to 0.10 or 0.001 to 0.09 wt %. The concentration of the phenyl-alpha-naphthylamine in the liquid additive can be determined by gas chromatography.

The phenyl-alpha-naphthylamine can be illustrated by the following formula (2)

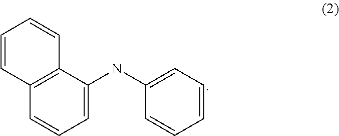 [SMILES: c1ccc(Nc2cccc3ccccc23)cc1]

Typically, hydrogen atoms are not always displayed in chemical formula. Accordingly, the phenyl-alpha-naphthylamine can also be illustrated by one the two following formula with hydrogen atoms included:

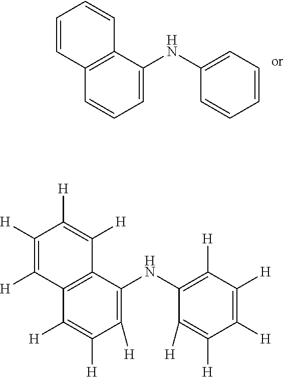 [SMILES: c1ccc(Nc2cccc3ccccc23)cc1 ; c1ccc(Nc2cccc3ccccc23)cc1]

The concentration of the olefin in the liquid additive can be below 1, 0.5, 0.3, 0.2, 0.1 or 0.05 wt %. The concentration of the olefin in the liquid additive can be determined by gas chromatography.

The liquid additive is usually free of an organic solvent. The concentration of the organic solvent in the liquid additive can be below 5, 3, 1, 0.5, 0.3, 0.2, 0.1 or 0.05 wt %.

The liquid additive is usually free of water. The concentration of water in the liquid additive can be below 5, 3, 1, 0.5, 0.3, 0.2, 0.1 or 0.05 wt %.

The liquid additive is usually a liquid at 21° C. The liquid additive may have a melting point of below 10° C., preferably below 0° C.

The liquid additive may have a reduced aquatic toxicity. A reduced aquatic toxicity can be determined by comparison of the liquid additive with a comparative amine of the formula (1), which contains 5 wt % of phenyl-alpha-naphthylamine.

The aquatic toxicity can be a short-term aquatic toxicity to fish (e.g. Oncorhynchus mykiss or Lepomis macrochirus), a long-term aquatic toxicity to fish (e.g. Oncorhynchus mykiss or Lepomis macrochirus), a short-term aquatic toxicity to aquatic invertebrates (e.g. Daphnia magna), or a long-term aquatic toxicity to aquatic invertebrates (e.g. Daphnia magna).

In one form the aquatic toxicity is the short-term aquatic toxicity to fish, which can be determined as LC50 (96 h) value in freshwater as semistatic test with Oncorhynchus mykiss. In another form the aquatic toxicity is the short-term aquatic toxicity to fish, which can be determined as LC50 (96 h) value in freshwater as semistatic test with Lepomis macrochirus.

In another form the aquatic toxicity is the long-term aquatic toxicity to aquatic invertebrates, which can be determined as NOEC (21 d) value based on reproduction in freshwater as semistatic test with Daphnia magna.

In another preferred form the aquatic toxicity is the long-term aquatic toxicity to aquatic invertebrates, which can be determined as LOEC (21 d) value based on reproduction in freshwater with Daphnia magna according to OECD guideline 202.

In another form the aquatic toxicity is the toxicity to freshwater aquatic plants of the genus Lemna (duckweed), which can be determined according to OECD guideline 221.

The olefin is selected from linear or branched C₈-C₁₂ alkenes, such as linear or branched C₈, C₉, C₁₀, C₁₁, or C₁₂ alkenes. Mixtures of linear and branched C₈-C₁₂ alkenes are also possible.

Preferably, the olefin is selected from branched C₈-C₁₂ alkenes, such as branched C₈, C₉, C₁₀, C₁₁, or C₁₂ alkenes. The olefin which is selected from linear or branched C₈-C₁₂ alkenes may have a content of at least 90, 95, 98 or 99% of the linear and branched C₈-C₁₂ alkenes, which can be determined by gas chromatography.

Preferably, the olefin is a linear or branched (preferable branched) C₉ alkene, such as n-1-nonene, n-2-nonene, 1,3-dimethyl-2-heptene, or 4,6-dimethyl-1-heptene. Mixtures of linear and branched C₉ alkyls are also possible. The olefin which is selected from linear or branched C₉ alkenes may have a content of at least 90, 95, 98 or 99% of the linear and branched C₉ alkenes, which can be determined by gas chromatography.

In another preferred form the olefin is a C₉ alkene obtainable by oligomerization of propylene, e.g. commercially available as propylene trimer, trimerpropene, isononene or C₉ rich C₈-10-branched alkenes, CAS 97593-01-6 or CAS 68526-55-6. The olefin which is a C₉ alkene obtainable by oligomerization of propylene may have a content of at least 80, 84, 86, 88, or 90% of the C₉ alkenes, which can be determined by gas chromatography.

The olefin has usually an content of mono-unsaturated compounds of at least 90, 95, 98 or 99%, which can be determined by gas chromatography.

### Step a) Alkylation

The step of alkylating the phenyl-alpha-naphthylamine with the olefin is usually catalyzed by an acid catalyst. Suitable acid catalysts are proton donors (so-called Brønsted acids), electron acceptor compounds (so-called Lewis acids), cation exchanger resins, aluminosilicates or naturally occurring or modified layered silicates.

Suitable proton donors are salt-forming inorganic or organic acids, e.g. mineral acids such as hydrochloric acid, sulphuric acid or phosphoric acid, carboxylic acids, e.g. acetic acid, or sulphonic acids, e.g. methanesulphonic acid, benzenesulphonic acid or p-toluenesulphonic acid.

Suitable electron acceptor compounds are tin tetra-chloride, zinc chloride, aluminum chloride or boron trifluoride etherate.

Suitable cation exchanger resins are styrene-divinylbenzene copolymers containing sulpho acid groups as ion exchanger function, e.g. the known products Amberlite® and Amberlyst®, e.g. AMBERLITE 200, or Dowex® 50, perfluorinated ion exchanger resins, e.g. Nafion® H, of DuPont, or other superacid ion exchanger resins.

Suitable aluminosilicates are amorphous aluminum silicates which contain about 10-30% of aluminum oxide and about 70-90% of silicon dioxide, e.g. aluminum silicate HA-HPV® of Ketjen (Akzo), or crystalline aluminum silicates, e.g. so-called zeolites, which are used as inorganic cation exchangers, as so-called molecular sieves or in the petrochemistry as so-called cracking catalysts, e.g. faujasites, e.g. Zeolite X, e.g. 13X (union Carbide) or SZ-9 (Grace), Zeolite Y, e.g. LZ-82 (Union Carbide), Ultrastable Y Zeolite, e.g. Octacat (Grace), mordenites. e.g. Zeolon 900H® (Norton), or Zeolite Beta, e.g. H-BEA (Südchemie), or Zeolite ZSM-12® (Mobil Oil).

Suitable naturally occurring layered silicates are also called acid earths or clays and are e.g. montmorillonites which are activated e.g. with mineral acids, such as sulphuric acid and/or hydrochloric acid, and which preferably have a moisture content of less than 10%, preferably of less than 5%, for example so-called earths or clays of the Fuller type, e.g. the types commercially available under the name Fulcat® (Rockwood Additives), e.g. the types Fulcat 22 B, 220, 230 and 240 (clays activated with sulphuric acid), Fulmont® (Rockwood Additives), e.g. the types XMP-4, XMP-3, or acid clays of the types K5, K10, K20 and K30 (activated with hydrochloric acid), KS and KSF (activated with sulphuric acid) or KSFO (activated with hydrochloric acid and sulphuric acid), of Südchemie, and also clays based on bentonite, e.g. products of the types Filtrol® or Retrol®, e.g. F-13, F-20 (Engelhard Corp.).

Preferably, the acid catalyst is Fulcat® 22 B, an acid-activated montmorrillonite.

Modified layered silicates are also called pillared clays and are derived from the above-described naturally occurring layered silicates, containing between the silicate layers oxides of e.g. zirconium, iron, zinc, nickel, chromium, cobalt or magnesium, or rare earth elements. Suitable modified layered silicates are the products Envirocat® EPZ-10, EPZG or EPIC produced by Contract Chemicals.

The acid catalyst can be added, for example, in an amount of 1-50, preferably of 5-25, highly preferred of 5-20, percent by weight in respect to the weight amounts of the amine reactants employed or, in the event that a so-called Brønsted acid or Lewis acid is used, in an amount of 0.002 to 10 mol %, preferably of 0.1 to 5.0 mol % in respect to the weight amounts of the amine.

For the alkylating step a molar ratio of the phenyl-alpha-naphthylamine to the olefin can be used in the range from 1:0.8 to 1:20, from 1:1 to 1:15, from 1:2 to 1:10, from 1:3 to 1:10, from 1:4 to 1:10, or from 1:5 to 1:10.

The alkylating of phenyl-alpha-naphthylamine can be made with or without solvent, preferably without solvent. If a solvent is used, it should be inert under the given reaction conditions and should have a suitably high boiling temperature. Suitable solvents are optionally halogenated hydrocarbons, polar aprotic solvents, liquid amides and alcohols. Solvents to be mentioned as examples are: petroleum ether fractions, preferably higher boiling ones, toluene, mesitylene, dichlorobenzene, tetrahydrofuran (THF), dimethylformamide (DMF), dimethylacet amide (DMA), hexamethylphosphoric acid triamide (HMPTA), glymes and diglymes, dime-thylsulphoxide (DMSO), tetramethylurea (TMU), higher alcohols, such as butanol or ethylene glycol.

The alkylating of phenyl-alpha-naphthylamine can be made at a reaction temperature of at least 100, 120 or 140° C., and up to 250, 210 or 180° C.

The alkylation can be carried out by introducing the starting materials and the acid clays, as the catalyst, into a suitable reaction vessel and by heating to the temperatures specified. In an alternative process variant, the olefin can be added to the reaction mixture later. The feed time of the olefin is usually 0.5-24 hours. After the feeding of the olefin to the phenyl-alpha-naphthylamine the reaction mixture can be kept at the reaction temperature for 0.5-24 hours, e.g. at least 2, 4, 6, 8, 10, 12, 14, 16, or 18 hours. The alkylation is preferably carried out without the addition of organic solvents.

The progress of the alkylation can be followed, e.g. by gas chromatography. Usually, the amount of the phenyl-alpha-naphthylamine is below 20, 15, 10, or 5% based on the total amount of phenyl-alpha-naphthylamine and alkylated phenyl-alpha-naphthylamine.

The alkylation is preferably carried out under ambient pressure. Alkylation at elevated pressures is possible, for example in an autoclave under a pressure of from 1 to 10 bar absolute pressure.

The acid catalyst can be present in dissolved form or as solid. The acid catalysts used in the alkylation can be removed from the reaction mixture by filtration, centrifugation, decanting, extraction, precipitation, neutralizing, evaporation or distillation. The solid acid catalysts used in the alkylation can be removed from the reaction mixture by filtration, centrifugation or decanting. The dissolved acid catalysts used in the alkylation can be removed from the reaction mixture by extraction, precipitation, neutralizing, evaporation or distillation. The acid catalysts are typically re-usable. After removing the solid acid catalysts it can be washed with the olefin before recycling it, e.g. in the alkylating step.

At the end of the alkylating step a) the first raw product is obtained, which comprises the amine of the formula (1), and typically the unreacted olefin and the unreacted phenyl-alpha-naphthylamine. The amounts of the unreacted olefin and the unreacted phenyl-alpha-naphthylamine in the first raw product usually depend on the reaction conditions in the step a). The amount of the unreacted olefin in the first raw product can be at least 5, 10, 20, 30, 50, or 70 wt %. The amount of the unreacted phenyl-alpha-naphthylamine in the first raw product can be at least 0.3, 0.5, 0.7, 01, 2, 3, 4, 5, or 10 wt %.

### Step b) Distilling Olefin

The step b) is distilling off the unreacted olefin from the first raw product to obtain a second raw product. The unreacted olefin is usually resulting from the molar excess of the olefin over the phenyl-alpha-naphthylamine used in the alkylation step a).

The distillation conditions are usually adapted to the boiling point of the olefin. Typically, the distillation of the olefin is achieved at elevated temperatures and/or in vacuum. For example in step b) the first raw product can be heated in the sump to at least 50, 80, 100, 110, 120, 130, 140, or 150° C. at vacuum of below 1000, 800, 700, 600, 500, 400, 300, 200, or 100 mbar.

Preferably, in step b) the first raw product can be heated to 80 to 280° C. at vacuum of 1000 to 20 mbar, or 100 to 180° C. at vacuum of 600 to 100 mbar.

The distillation in step b) can be made at a reflux ratio in the range from 10:1 to 1:2, preferably from 5:1 to 1:1.5, and in particular 3:1 to 1:1.1, where the reflux ratio is usually calculated as parts recycle to the top of the distillation column: parts collected in the distillate receiver.

The second raw product obtained in step b) usually contains up to 10, 5, 4, 3, 2, 1, or 0.5 wt % of the olefin. The amount of olefin can be determined by gas chromatography.

When the distillation in step b) is made batch wise, it may take from 0.5 to 24 hours, or 1 to 18 hours, or 2 to 12 hours. The distillation can also be made as continuous process, semi-batch process, or a combination of both (e.g. when two distiller setups are used).

The distillation in step b) can be made is a usual distillation apparatus, e.g. a distillation vessel, a distillation apparatus with packed columns, tray columns, columns with beds of packing elements. The material of construction can be stainless steel, glass, glass-lined, or fluorpolymer.

The distilled off unreacted olefin can be recycled, for example in the alkylation of step a). The unreacted olefin may be collected, e.g. on site in tank, and then added to the alkylation. The unreacted olefin may also be forwarded directly and added continuously to the alkylation.

The method for manufacturing the liquid additive comprises optionally a further step of d) recycling the distilled off unreacted olefin from the step b) in the alkylation of step a). The unreacted olefin may be collected in tanks before recycling it in the alkylation of step a).

### Step c) Distilling Phenyl-Alpha-Naphthylamine

The step c) is distilling off the unreacted phenyl-alpha-naphthylamine from the second raw product to obtain the liquid additive.

Typically, the distillation of the phenyl-alpha-naphthylamine is achieved at elevated temperatures and/or in vacuum. For example in step c) the second raw product can be heated in the sump to at least 100, 120, 140, 160, 180, 200, 210, 220, 230, 240 or 250° C. at a vacuum of below 400, 300, 200, 100, 80, 60, 50, 40, 30, 20 or 10 mbar. In a preferred form in step c) the second raw product can be heated in the sump to at least 100° C. at a vacuum of below 400 mbar. In another preferred form in step c) the second raw product can be heated in the sump to at least 150° C. at a vacuum of below 200 mbar. In another preferred form in step c) the second raw product can be heated in the sump to at least 200° C. at a vacuum of below 100 mbar.

Preferably, in step c) the second raw product can be heated to 100 to 300° C. at vacuum of 400 to 1 mbar, or 180 to 300° C. at vacuum of below 200 mbar, or 210 to 280° C. at vacuum of below 150 mbar.

In another preferred form of step c) the second raw product can be heated to 120 to 270° C. at vacuum of 400 to 1 mbar, or 150 to 300° C. at vacuum of 200 to 5 mbar, or 210 to 280° C. at vacuum of 150 to 5 mbar.

The distillation in step c) can be made at a reflux ratio in the range from 1:1 to 1:20, preferably from 1:3 to 1:10, and in particular 1:4 to 1:8, where the reflux ratio is usually calculated as parts recycle to the top of the distillation column: parts collected in the distillate receiver.

When the distillation is made batch wise, it may take from 0.5 to 24 hours, or 1 to 18 hours, or 2 to 12 hours. The distillation can also be made as continuous process or semi-batch process.

The distillation in step c) can be made in a distillation column with at least 3, 4, 5, or 10 theoretical separation stages.

The distillation in steps b) and c) can be performed in the same distiller or in two different distillers.

The distillation in step c) can be made is a usual distillation apparatus, e.g. a distillation vessel, a distillation apparatus with packed columns, tray columns, columns with beds of packing elements.

The distilled off phenyl-alpha-naphthylamine obtained in the step c) can be recycled in the alkylation of the step a).

The liquid additive obtained from step c) can be cooled down, e.g. to below 100° C., and then packed, e.g. in drums.

The liquid additive obtained from step c) can be used in lubricants without further purification steps.

The method for manufacturing the liquid additive is usually free of a crystallization step, such as crystallization of the phenyl-alpha-naphthylamine, crystallization of the olefin, crystallization of the amine of the formula (1), or crystallization of any reaction by-products.

The method for manufacturing the liquid additive comprises the steps a), b) and c), which are usually made in the alphabetic order.

### Use as Lubricant

The present invention also relates to a use of the liquid additive in lubricants, e.g. the use as lubricant additive.

A method for lubricating moving surfaces may comprise the steps a), b) and c) to obtain the liquid additive, followed by a step of blending of the liquid additive with a base oil to obtain an formulated lubricant, and followed by a step of contacting the moving surfaces with the formulated lubricant.

The lubricant is usually composition which is capable of reducing friction between surfaces (preferably metal surfaces), such as surfaces of mechanical devices. A mechanical device may be a mechanism consisting of a device that works on mechanical principles. Suitable mechanical device are bearings, gears, joints and guidances. The mechanical device may be operated at temperatures in the range of −40° C. to 180° C. Lubricants are usually specifically formulated for virtually every type of machine and manufacturing process. The type and concentration of base oils and/or lubricant additives used for these lubricants may be selected based on the requirements of the machinery or process being lubricated, the quality required by the builders and the users of the machinery, and the government regulation. Typically, each lubricant has a unique set of performance requirements. In addition to proper lubrication of the machine or process, these requirements may include maintenance of the quality of the lubricant itself, as well as the effect of the lubricant's use and disposal on energy use, the quality of the environment, and on the health of the user.

Typical lubricants are automotive lubricants (e.g. gasoline engine oils, diesel engine oils, gas engine oils, gas turbine oils, automatic transmission fluids, gear oils) and industrial lubricants (e.g. industrial gear oils, pneumatic tool lubricating oil, high temperature oil, gas compressor oil, hydraulic fluids, metalworking fluids).

Examples for lubricants are axel lubrication, medium and heavy duty engine oils, industrial engine oils, marine engine oils, automotive engine oils, crankshaft oils, compressor oils, refrigerator oils, hydrocarbon compressor oils, very low-temperature lubricating oils and fats, high temperature lubricating oils and fats, wire rope lubricants, textile machine oils, refrigerator oils, aviation and aerospace lubricants, aviation turbine oils, transmission oils, gas turbine oils, spindle oils, spin oils, traction fluids, transmission oils, plastic transmission oils, passenger car transmission oils, truck transmission oils, industrial transmission oils, industrial gear oils, insulating oils, instrument oils, brake fluids, transmission liquids, shock absorber oils, heat distribution medium oils, transformer oils, fats, chain oils, minimum quantity lubricants for metalworking operations, oil to the warm and cold working, oil for water-based metalworking liquids, oil for neat oil metalworking fluids, oil for semi-synthetic metalworking fluids, oil for synthetic metalworking fluids, drilling detergents for the soil exploration, hydraulic oils, in biodegradable lubricants or lubricating greases or waxes, chain saw oils, release agents, molding fluids, gun, pistol and rifle lubricants or watch lubricants and food grade approved lubricants.

The lubricant is usually a lubricating liquid, lubricating oil or lubricating grease.

The lubricant may comprise at least 0.1 wt %, preferably at least 0.5 wt % and in particular at least 1 wt % of the liquid additive.

In another form the lubricant may comprise 0.1-20 wt %, preferably 0.5-15 wt % and in particular at least 1-10 wt % of the liquid additive.

The lubricant comprises the liquid additive and a base oil. The lubricant may comprise at least 25, 30, 35, 40, 45, 50, 55, 60, 65, 70, 75 or 80 wt % of the base oil. The lubricant may comprise up to 99.9, 98, 95, 90, 80, 70, or 50 wt % of the base oil.

The base oil may selected from the group consisting of mineral oils (Group I, II or III oils), polyalphaolefins (Group IV oils), polymerized and interpolymerized olefins, alkyl naphthalenes, alkylene oxide polymers, silicone oils, phosphate esters and carboxylic acid esters (Group V oils). Preferably, the base oil is selected from Group I, Group II, Group III base oils according to the definition of the API, or mixtures thereof. Definitions for the base oils are the same as those found in the American Petroleum Institute (API) publication “Engine Oil Licensing and Certification System”, Industry Services Department, Fourteenth Edition, December 1996, Addendum 1, December 1998. Said publication categorizes base oils as follows:
a) Group I base oils contain less than 90 percent saturates (ASTM D 2007) and/or greater than 0.03 percent sulfur (ASTM D 2622) and have a viscosity index (ASTM D 2270) greater than or equal to 80 and less than 120.
b) Group II base oils contain greater than or equal to 90 percent saturates and less than or equal to 0.03 percent sulfur and have a viscosity index greater than or equal to 80 and less than 120.
c) Group III base oils contain greater than or equal to 90 percent saturates and less than or equal to 0.03 percent sulfur and have a viscosity index greater than or equal to 120.
d) Group IV base oils contain polyalphaolefins. Polyalphaolefins (PAO) include known PAO materials which typically comprise relatively low molecular weight hydrogenated polymers or oligomers of alphaolefins which include but are not limited to C2 to about C32 alpha-olefins with the C8 to about C16 alphaolefins, such as 1-octene, 1-decene, 1-dodecene and the like being preferred. The preferred polyalphaolefins are poly-1-octene, poly-1-de-cene, and poly-1-dodecene.
e) Group V base oils contain any base oils not described by Groups I to IV. Examples of Group V base oils include alkyl naphthalenes, alkylene oxide polymers, silicone oils, and phosphate esters.

Synthetic base oils include hydrocarbon oils and halo-substituted hydrocarbon oils such as polymerized and interpolymerized olefins (e.g., polypropylenes, propylene-isobutylene copolymers, chlorinated polybutylenes, poly(l-hexenes), poly(l-octenes), poly(l-decenes)); alkylbenzenes (e.g., dodecylbenzenes, tetradecylbenzenes, dinonylbenzenes, di(2-ethylhexyl)benzenes); poly-phenyls (e.g., biphenyls, terphenyls, alkylated polyphenols); and alkylated diphenyl ethers and alkylated diphenyl sulfides and derivative, analogs and homologs thereof.

Alkylene oxide polymers and interpolymers and derivatives thereof where the terminal hydroxyl groups have been modified by esterification, etherification, etc., constitute another class of known synthetic base oils. These are exemplified by polyoxyalkylene polymers prepared by polymerization of ethylene oxide or propylene oxide, and the alkyl and aryl ethers of polyoxyalkylene polymers (e.g., methyl-polyiso-propylene glycol ether having a molecular weight of 1000 or diphenyl ether of polyethylene glycol having a molecular weight of 1000 to 1500); and mono- and polycarboxylic esters thereof, for example, the acetic acid esters, mixed C3-C8 fatty acid esters and C13 oxo acid diester of tetraethylene glycol.

Silicon-based oils such as the polyalkyl-, polyaryl-, polyalkoxy- or polyaryloxysilicone oils and silicate oils comprise another useful class of synthetic base oils; such base oils include tetra-ethyl silicate, tetraisopropyl silicate, tetra-(2-ethylhexyl) silicate, tetra-(4-methyl-2-ethyl-hexyl) silicate, tetra-(p-tert-butyl-phenyl) silicate, hexa-(4-methyl-2-ethylhexyl) disiloxane, poly(methyl) siloxanes and poly(methylphenyl) siloxanes. Other synthetic base oils include liquid esters of phosphorous-containing acids (e.g., tricresyl phosphate, trioctyl phosphate, diethyl ester of decylphosphonic acid) and polymeric tetrahydrofurans.

The lubricant usually further comprises a lubricant additive. Suitable lubricant additives may be selected from viscosity index improvers, polymeric thickeners, antioxidants, corrosion inhibitors, detergents, dispersants, anti-foam agents, dyes, wear protection additives, extreme pressure additives (EP additives), anti-wear additives (AW additives), friction modifiers, metal deactivators, pour point depressants.

The viscosity index improvers include high molecular weight polymers that increase the relative viscosity of an oil at high temperatures more than they do at low temperatures. Viscosity index improvers include polyacrylates, polymethacrylates, alkylmethacrylates, vinylpyrrolidone/meth-acrylate copolymers, poly vinylpyrrolidones, polybutenes, olefin copolymers such as an ethylene-propylene copolymer or a styrene-butadiene copolymer or polyalkene such as PIB, styrene/acrylate copolymers and polyethers, and combinations thereof. The most common VI improvers are methacrylate polymers and copolymers, acrylate polymers, olefin polymers and copolymers, and styrenebutadiene copolymers. Other examples of the viscosity index improver include polymethacrylate, polyisobutylene, alpha-olefin polymers, alpha-olefin copolymers (e.g., an ethylenepropylene copolymer), polyalkylstyrene, phenol condensates, naphthalene condensates, a styrenebutadiene copolymer and the like. Of these, polymethacrylate having a number average molecular weight of 10000 to 300000, and alpha-olefin polymers or alpha-olefin copolymers having a number average molecular weight of 1000 to 30000, particularly ethylene-alpha-olefin copolymers having a number average molecular weight of 1000 to 10000 are preferred. The viscosity index increasing agents can be added and used individually or in the form of mixtures, conveniently in an amount within the range of from ≥0.05 to ≤20.0% by weight, in relation to the weight of the base stock.

Suitable (polymeric) thickeners include, but are not limited to, polyisobutenes (PIB), oligomeric co-polymers (OCPs), polymethacrylates (PMAs), copolymers of styrene and butadiene, or high viscosity esters (complex esters).

Antioxidants include phenolic antioxidants such as hindered phenolic antioxidants or non-phenolic oxidation inhibitors.

Useful phenolic antioxidants include hindered phenols. These phenolic antioxidants may be ashless (metal-free) phenolic compounds or neutral or basic metal salts of certain phenolic compounds. Typical phenolic antioxidant compounds are the hindered phenolics which are the ones which contain a sterically hindered hydroxyl group, and these include those derivatives of dihydroxy aryl compounds in which the hydroxyl groups are in the o- or p-position to each other. Typical phenolic antioxidants include the hindered phenols substituted with alkyl groups having 6 carbon atoms or more and the alkylene coupled derivatives of these hindered phenols. Examples of phenolic materials of this type 2-t-butyl-4-heptyl phenol; 2-t-butyl-4-octyl phenol; 2-t-butyl-4-dodecyl phenol; 2,6-di-t-butyl-4-heptyl phenol; 2,6-di-t-butyl-4-dodecyl phenol; 2-methyl-6-t-butyl-4-heptyl phenol; and 2-methyl-6-t-butyl-4-dodecyl phenol. Other useful hindered mono-phenolic antioxidants may include for example hindered 2,6-di-alkyl-phenolic propionic ester derivatives. Bis-phenolic antioxidants may also be used in combination with the present invention. Examples of ortho-coupled phenols include: 2,2′-bis(4-heptyl-6-t-butyl-phenol); 2,2′-bis(4-octyl-6-t-butyl-phenol); and 2,2′-bis(4-dodecyl-6-t-butyl-phenol). Para-coupled bisphenols include for example 4,4′-bis(2,6-di-t-butyl phenol) and 4,4′-methylene-bis(2,6-di-t-butyl phenol).

Non-phenolic oxidation inhibitors which may be used include aromatic amine antioxidants and these may be used either as such or in combination with phenolics. Typical examples of non-phenolic antioxidants include: alkylated and non-alkylated aromatic amines such as aromatic monoamines of the formula R⁸R⁹R¹⁰N, where R⁸ is an aliphatic, aromatic or substituted aromatic group, R⁹ is an aromatic or a substituted aromatic group, and R¹⁰ is H, alkyl, aryl or R¹¹S(O)ₓR¹², where R¹¹ is an alkylene, alkenylene, or aralkylene group, R¹² is a higher alkyl group, or an alkenyl, aryl, or alkaryl group, and x is 0, 1 or 2. The aliphatic group R⁸ may contain from 1 to about 20 carbon atoms, and preferably contains from about 6 to 12 carbon atoms. The aliphatic group is a saturated aliphatic group. Preferably, both R⁸ and R⁹ are aromatic or substituted aromatic groups, and the aromatic group may be a fused ring aromatic group such as naphthyl. Aromatic groups R⁸ and R⁹ may be joined together with other groups such as S.

Typical aromatic amines antioxidants have alkyl substituent groups of at least about 6 carbon atoms. Examples of aliphatic groups include hexyl, heptyl, octyl, nonyl, and decyl. Generally, the aliphatic groups will not contain more than about 14 carbon atoms. The general types of amine antioxidants useful in the present compositions include diphenylamines, phenyl naphthylamines, phenothiazines, imidodibenzyls and diphenyl phenylene diamines. Mixtures of two or more aromatic amines are also useful. Polymeric amine antioxidants can also be used.

Particular examples of aromatic amine antioxidants useful in the present invention include: p,p′-dioctyldiphenylamine; t-octylphenyl-alpha-naphthylamine; phenyl-alphanaphthylamine; and p-octylphenyl-alpha-naphthylamine. Sulfurized alkyl phenols and alkali or alkaline earth metal salts thereof also are useful antioxidants.

Corrosion inhibitors may include various oxygen-, nitrogen-, sulfur-, and phosphorus-containing materials, and may include metal-containing compounds (salts, organometallics, etc.) and nonmetal-containing or ashless materials. Corrosion inhibitors may include, but are not limited to, additive types such as, for example, hydrocarbyl-, aryl-, alkyl-, arylalkyl-, and alkylaryl-versions of detergents (neutral, overbased), sulfonates, phenates, salicylates, alcoholates, carboxylates, salixarates, phosphites, phosphates, thiophosphates, amines, amine salts, amine phosphoric acid salts, amine sulfonic acid salts, alkoxylated amines, etheramines, polyether-amines, amides, imides, azoles, diazoles, triazoles, benzotriazoles, benzothiadoles, mercapto-benzothiazoles, tolyltriazoles (TTZ-type), heterocyclic amines, heterocyclic sulfides, thiazoles, thiadiazoles, mercaptothiadiazoles, dimercaptothiadiazoles (DMTD-type), imidazoles, benzimidazoles, dithiobenzimidazoles, imidazolines, oxazolines, Mannich reactions products, glycidyl ethers, anhydrides, carbamates, thiocarbamates, dithiocarbamates, polyglycols, etc., or mixtures thereof.

Detergents include cleaning agents that adhere to dirt particles, preventing them from attaching to critical surfaces. Detergents may also adhere to the metal surface itself to keep it clean and prevent corrosion from occurring. Detergents include calcium alkylsalicylates, calcium alkylphenates and calcium alkarylsulfonates with alternate metal ions used such as magnesium, barium, or sodium. Examples of the cleaning and dispersing agents which can be used include metal-based detergents such as the neutral and basic alkaline earth metal sulphonates, alka-line earth metal phenates and alkaline earth metal salicylates alkenylsuccinimide and alkenyl-succinimide esters and their borohydrides, phenates, salienius complex detergents and ashless dispersing agents which have been modified with sulphur compounds. These agents can be added and used individually or in the form of mixtures, conveniently in an amount within the range of from ≥0.01 to ≤1.0% by weight in relation to the weight of the base stock; these can also be high total base number (TBN), low TBN, or mixtures of high/low TBN.

Dispersants are lubricant additives that help to prevent sludge, varnish and other deposits from forming on critical surfaces. The dispersant may be a succinimide dispersant (for example N-substituted long chain alkenyl succinimides), a Mannich dispersant, an ester-containing dispersant, a condensation product of a fatty hydrocarbyl monocarboxylic acylating agent with an amine or ammonia, an alkyl amino phenol dispersant, a hydrocarbyl-amine dispersant, a polyether dispersant or a polyetheramine dispersant. In one embodiment, the succinimide dispersant includes a polyisobutylene-substituted succinimide, wherein the polyisobutylene from which the dispersant is derived may have a number average molecular weight of about 400 to about 5000, or of about 950 to about 1600. In one embodiment, the dispersant includes a borated dispersant. Typically, the borated dispersant includes a succinimide dispersant including a polyisobutylene succinimide, wherein the polyisobutylene from which the dispersant is derived may have a number average molecular weight of about 400 to about 5000. Borated dispersants are described in more detail above within the extreme pressure agent description.

Anti-foam agents may be selected from silicones, polyacrylates, and the like. The amount of anti-foam agent in the lubricant compositions described herein may range from ≥0.001 wt.-% to ≤0.1 wt.-% based on the total weight of the formulation. As a further example, an anti-foam agent may be present in an amount from about 0.004 wt.-% to about 0.008 wt.-%.

Suitable extreme pressure agent is a sulfur-containing compound. In one embodiment, the sulfur-containing compound may be a sulfurised olefin, a polysulfide, or mixtures thereof. Examples of the sulfurised olefin include a sulfurised olefin derived from propylene, isobutylene, pentene; an organic sulfide and/or polysulfide including benzyldisulfide; bis-(chlorobenzyl) disulfide; dibutyl tetrasulfide; di-tertiary butyl polysulfide; and sulfurised methyl ester of oleic acid, a sulfurised alkylphenol, a sulfurised dipentene, a sulfurised terpene, a sulfurised Diels-Alder adduct, an alkyl sulphenyl N′N-dialkyl dithiocarbamates; or mixtures thereof. In one embodiment, the sulfurised olefin includes a sulfurised olefin derived from propylene, isobutylene, pentene or mixtures thereof. In one embodiment the extreme pressure additive sulfur-containing compound includes a dimercaptothiadiazole or derivative, or mixtures thereof. Examples of the dimercaptothiadiazole include compounds such as 2,5-dimercapto-1,3,4-thiadiazole or a hydrocarbyl-substituted 2,5-dimercapto-1,3,4-thiadiazole, or oligomers thereof. The oligomers of hydrocarbyl-substituted 2,5-dimercapto-1,3,4-thiadiazole typically form by forming a sulfur-sulfur bond between 2,5-dimercapto-1,3,4-thiadiazole units to form derivatives or oligomers of two or more of said thiadiazole units. Suitable 2,5-dimercapto-1,3,4-thiadiazole derived compounds include for example 2,5-bis(tert-nonyldithio)-1,3,4-thiadiazole or 2-tert-nonyldithio-5-mercapto-1,3,4-thiadiazole. The number of carbon atoms on the hydrocarbyl substituents of the hydrocarbyl-substituted 2,5-dimercapto-1,3,4-thiadiazole typically include 1 to 30, or 2 to 20, or 3 to 16. Extreme pressure additives include compounds containing boron and/or sulfur and/or phosphorus. The extreme pressure agent may be present in the lubricant compositions at 0 wt.-% to about 20 wt.-%, or at about 0.05 wt.-% to about 10.0 wt.-%, or at about 0.1 wt.-% to about 8 wt.-% of the lubricant composition.

Examples of anti-wear additives include organo borates, organo phosphites such as didodecyl phosphite, organic sulfur-containing compounds such as sulfurized sperm oil or sulfurized terpenes, zinc dialkyl dithiophosphates, zinc diaryl dithiophosphates, phosphosulfurized hydrocarbons and any combinations thereof.

Friction modifiers may include metal-containing compounds or materials as well as ashless compounds or materials, or mixtures thereof. Metal-containing friction modifiers include metal salts or metal-ligand complexes where the metals may include alkali, alkaline earth, or transition group metals. Such metal-containing friction modifiers may also have low-ash characteristics. Transition metals may include Mo, Sb, Sn, Fe, Cu, Zn, and others. Ligands may include hydrocarbyl derivative of alcohols, polyols, glycerols, partial ester glycerols, thiols, carboxylates, carbamates, thiocarbamates, dithiocarbamates, phosphates, thiophosphates, dithiophosphates, amides, imides, amines, thiazoles, thiadiazoles, dithiazoles, diazoles, triazoles, and other polar molecular functional groups containing effective amounts of O, N, S, or P, individually or in combination. In particular, Mo-containing compounds can be particularly effective such as for example Mo-dithiocarbamates, Mo (DTC), Mo-dithiophosphates, Mo (DTP), Mo-amines, Mo (Am), Mo-alcoholates, Mo-alcohol-amides, and the like.

Ashless friction modifiers may also include lubricant materials that contain effective amounts of polar groups, for example, hydroxyl-containing hydrocarbyl base oils, glycerides, partial glycerides, glyceride derivatives, and the like. Polar groups in friction modifiers may include hydrocarbyl groups containing effective amounts of O, N, S, or P, individually or in combination. Other friction modifiers that may be particularly effective include, for example, salts (both ash-containing and ashless derivatives) of fatty acids, fatty alcohols, fatty amides, fatty esters, hydroxyl-containing carboxylates, and comparable synthetic long-chain hydrocarbyl acids, alcohols, amides, esters, hydroxy carboxylates, and the like. In some instances, fatty organic acids, fatty amines, and sulfurized fatty acids may be used as suitable friction modifiers. Examples of friction modifiers include fatty acid esters and amides, organo molybdenum compounds, molybdenum dialkylthiocarbamates and molybdenum dialkyl dithiophosphates.

Suitable metal deactivators include benzotriazoles and derivatives thereof, for example 4- or 5-alkylbenzotriazoles (e.g. triazole) and derivatives thereof, 4,5,6,7-tetrahydrobenzotriazole and 5,5′-methylenebisbenzotriazole; Mannich bases of benzotriazole or triazole, e.g. 1-[bis(2-ethyl-hexyl) aminomethyl) triazole and 1-[bis(2-ethylhexyl) aminomethyl)benzotriazole; and alkoxy-alkylbenzotriazoles such as 1-(nonyloxymethyl)benzotriazole, 1-(1-butoxyethyl)benzotriazole and 1-(1-cyclohexyloxybutyl) triazole, and combinations thereof. Additional non-limiting examples of the one or more metal deactivators include 1,2,4-triazoles and derivatives thereof, for example 3-alkyl (or aryl)-1,2,4-triazoles, and Mannich bases of 1,2,4-triazoles, such as 1-[bis(2-ethylhexyl) aminomethyl-1,2,4-triazole; alkoxyalkyl-1,2,4-triazoles such as 1-(1-butoxyethyl)-1,2,4-triazole; and acylated 3-amino-1,2,4-triazoles, imidazole derivatives, for example 4,4′-methylenebis(2-undecyl-5-methylimidazole) and bis[(N-methyl) imidazol-2-yl]car-binol octyl ether, and combinations thereof. Further non-limiting examples of the one or more metal deactivators include sulfur-containing heterocyclic compounds, for example 2-mercapto-benzothiazole, 2,5-dimercapto-1,3,4-thia-diazole and derivatives thereof; and 3,5-bis[di(2-ethylhexyl) aminomethyl]-1,3,4-thiadiazolin-2-one, and combinations thereof. Even further non-limiting examples of the one or more metal deactivators include amino compounds, for example salicylidenepropylenediamine, salicylami-noguanidine and salts thereof, and combinations thereof. The one or more metal deactivators are not particularly limited in amount in the composition but are typically present in an amount of from about 0.01 to about 0.1, from about 0.05 to about 0.01, or from about 0.07 to about 0.1, wt.-% based on the weight of the composition. Alternatively, the one or more metal deactivators may be present in amounts of less than about 0.1, of less than about 0.7, or less than about 0.5, wt.-% based on the weight of the composition.

Pour point depressants (PPD) include polymethacrylates, alkylated naphthalene derivatives, and combinations thereof. Commonly used additives such as alkylaromatic polymers and polymethacrylates are also useful for this purpose. Typically, the treat rates range from ≥0.001 wt.-% to ≤1.0 wt.-%, in relation to the weight of the base stock.

Demulsifiers include trialkyl phosphates, and various polymers and copolymers of ethylene glycol, ethylene oxide, propylene oxide, or mixtures thereof.

The invention also relates to the liquid additive comprising
an amine of the formula (1)

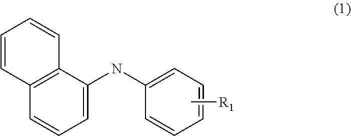

where R₁ is a linear or branched C₈-C₁₂ alkyl, and
phenyl-alpha-naphthylamine,
where the liquid additive comprises at least 95 wt % of the amine of the formula (1), and
where the concentration of the phenyl-alpha-naphthylamine in the liquid additive is below 0.2 wt %, preferably below 0.1 wt %.

The liquid additive is preferably obtainable by the method for manufacturing the liquid additive comprising the steps a), b) and c), and optionally d).

The liquid additive has preferably a reduced aquatic toxicity.

### EXAMPLES

### Raw Materials

Amine A: n-Phenyl-1-naphthylamine, purity >99.5%.
Olefin A: technical quality of trimerized propylene, >90 wt % C₉, olefin content >98% (ASTM D1319), density at 20° C. about 0.74 (ASTM D4052).
Fulcat® 22B: Acid activated Montmorillonite clay catalyst.

### Gas Chromatography

Samples (about 2% in toluene) were analyzed by gas chromatography on a DB-5 capillary (15 m length, 0.32 mm diameter) and FID detector. Temperature Program was started with a holding time at 60° C. for 2 minutes, then heating to 300° C. with 30° C./min and finally a holding time at 300° C. of 7 minutes. Typical retention times were 1.5 min for Olefin A, 14 min for Amine A, and 21 min for the alkylated Amine A.

### Example 1

Molten Amine A (1.0 mol equivalent) and the catalyst Fulcat® 22B (11.7 wt % based on the weight of Amine A) were loaded into a 2 liter stainless steel lab reactor and heated to 150° C. The Olefin A (4.0 mol equivalents) was added during several hours to the reactor. When the alkylation reaction was finished the reactor was cooled down to 100° C., the catalyst was filtered off and washed with Olefin A to obtain the first raw product.

The excess of unreacted Olefin A was distilled off from the first raw product in vacuum and the second raw product was obtained, which contained 11% of Amine A (based on the total amount of Amine 1 and alkylated Amine A). The distilled off Olefin A was recycled in further alkylation reactions.

The unreacted Amine A was distilled off from the second raw product at 210 to 250° C. at 100 to 150 mbar vacuum. The distilled off Amine A can be recycled for further alkylation reactions.

The final product contained 0.1% of Amine A and 99.9% alkylated Amine A as determined by gas chromatography. The final product was a clear red liquid.

### Example 2

Molten Amine A (1.0 mol equivalent) and the catalyst Fulcat® 22B (13.2 wt % based on the weight of Amine A) were loaded into a 2 liter stainless steel lab reactor and heated to 150° C. The Olefin A (4.0 mol equivalents) was added during several hours to the reactor. When the alkylation reaction was finished the reactor was cooled down to 100° C., the catalyst was filtered off and washed with Olefin A to obtain the first raw product.

The excess of unreacted Olefin A was distilled off from the first raw product in vacuum and the second raw product was obtained, which contained 6% of Amine A (based on the total amount of Amine 1 and alkylated Amine A). The distilled off Olefin A was recycled in further alkylation reactions.

The unreacted Amine A was distilled off from the second raw product at 210 to 250° C. at 100 to 150 mbar vacuum. The distilled off Amine A can be recycled for further alkylation reactions.

The final product contained 0.1% of Amine A and 99.9% alkylated Amine A as determined by gas chromatography. The final product was a clear red liquid.

### Example 3

The aquatic toxicity of samples which contained <0.1% of Amine A and >99.9% alkylated Amine A (as determined by gas chromatography) was tested according to OECD standards:

OECD Test 202 “Daphnia sp. Acute Immobilisation Test”

This test determined the acute toxicity to assess effects of chemicals towards daphnids. No toxic effects were found and the EC50 was below 0.223 μg/l. For comparison, commercially available Amine A safety data sheets show high aquatic toxic EC50 of 0.32 mg/l in the OECD Test 202.

OECD Test 221 “Lemna sp. Growth Inhibition Test”

This test determined the toxicity of substances to freshwater aquatic plants of the genus Lemna (duckweed). No toxic effects up to solubility limit were found.

1.-15. (canceled)

16. A method for manufacturing a liquid additive comprising an amine of the formula (1) 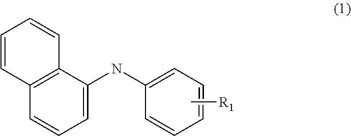
where R₁ is a linear or branched C₈-C₁₂ alkyl,
where the method comprises the steps of:
a) alkylating phenyl-alpha-naphthylamine with an olefin selected from linear or branched C₈-C₁₂ alkenes to obtain a first raw product comprising the amine of the formula (1);
b) distilling off unreacted olefin from the first raw product to obtain a second raw product; and
c) distilling off unreacted phenyl-alpha-naphthylamine from the second raw product to obtain the liquid additive; and
where in step a) a molar ratio of the phenyl-alpha-naphthylamine to the olefin is used in the range from 1:4 to 1:10; and
where the liquid additive comprises the phenyl-alpha-naphthylamine, and the concentration of the phenyl-alpha-naphthylamine in the liquid additive is below 0.2 wt %.

17. The method according to claim 16, where the liquid additive comprises at least 95 wt % of the amine of the formula (1).

18. The method according to claim 16, where the liquid additive comprises the phenyl-alpha-naphthylamine, and the concentration of the phenyl-alpha-naphthylamine in the liquid additive is below 0.1 wt %.

19. The method according to claim 16, where the olefin is selected from linear or branched C₉ alkenes.

20. The method according to claim 16, where in step a) a molar ratio of the phenyl-alpha-naphthylamine to the olefin is used in the range from 1:5 to 1:10.

21. The method according to claim 16, where in step c) the second raw product is be heated in the sump to at least 100° C. at a vacuum of below 400 mbar.

22. The method according to claim 16, where in step c) the second raw product is be heated in the sump to at least 200° C. at a vacuum of below 100 mbar.

23. The method according to claim 16, further comprising a step
d) recycling the distilled off unreacted olefin from the step b) in the alkylation of step a).

24. The method according to claim 16, where the method is free of a crystallization step.

25. The method according to claim 16, where the liquid additive has a melting point of below 10° C.

26. The method according to claim 16, where the liquid additive has a reduced aquatic toxicity.

27. A liquid additive comprising
an amine of the formula (1) 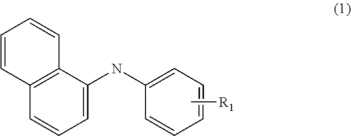
where R₁ is a linear or branched C₈-C₁₂ alkyl, and
phenyl-alpha-naphthylamine,
where the liquid additive comprises at least 95 wt % of the amine of the formula (1), and where the concentration of the phenyl-alpha-naphthylamine in the liquid additive is below 0.2 wt %.

28. A liquid additive comprising
an amine of the formula (1) 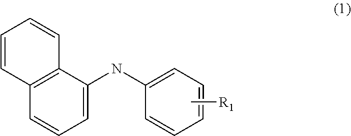
where R₁ is a linear or branched C₈-C₁₂ alkyl, and
phenyl-alpha-naphthylamine,
where the liquid additive comprises at least 95 wt % of the amine of the formula (1), and where the concentration of the phenyl-alpha-naphthylamine in the liquid additive is below 0.2 wt %, wherein the liquid additive is obtained by the method for manufacturing as defined in claim 16.

29. The liquid additive according to claim 27, where the liquid additive has a reduced aquatic toxicity.

30. A lubricant comprising the liquid additive as defined in claim 27.
